(19) United States
(12) Patent Application Publication
(10) Pub. No.: US 2024/0398670 A1
(43) Pub. Date: Dec. 5, 2024

(54) MEDICAL-TUBE AFFIXING DEVICE

(71) Applicant: Chien-Chung Su, Taichung City (TW)

(72) Inventors: CHIEN-CHUNG SU, Taichung City (TW); Hsin-Liang Han, Taichung City (TW)

(21) Appl. No.: 18/678,614

(22) Filed: May 30, 2024

(30) Foreign Application Priority Data
May 31, 2023 (TW) 112120406

(51) Int. Cl. A61J 15/00

(52) U.S. Cl. CPC A61J 15/0053

(57) ABSTRACT

A medical-tube affixing device includes an adhesive member, a base, and a clipping assembly. The adhesive member is for adhering to a patient at one side and for carrying the base at a reverse side. The base is for the clipping assembly to movably mount thereon. The clipping assembly is operatable to hold and position medical tubes with respect to the patient. With use of the disclosed medical-tube affixing device, medical tubes of different sizes can be easily and firmly attached to/detached from and positioned and/or repositioned, thereby improving working efficiency of healthcare workers.

### BACKGROUND OF THE INVENTION

### 1. Technical Field

The present invention relates to a medical-tube affixing device, and more particularly to a medical-tube affixing device developed primarily for extracorporeal membrane oxygenation (ECMO) in medical applications.

### 2. Description of Related Art

Tubes and pipes are used in various medical treatments, and may be known as intravenous drips, cannulas, urinary catheters, oxygen hoses, thoracic ducts, nasogastric tubes, tracheostomy tubes, and so on. Some of these tubes are for long use and have to be well fixed to patients in order to prevent tubes from unintentionally loosing or falling or entangling with patients' limbs.

Similarly, some patients may have to use indwelling catheters for long periods due to their conditions. For example, for patients with long term mechanical ventilation, patients after surgical operations, and parents with some catastrophic illness, doctors may instruct them to intermittently suspend use of the tubes according to their conditions instead of removing the tubes. In such a rest period, it is necessary to properly fix the tubes. Otherwise, once the tubes are bended or compressed, or even coming off, the patients are subject to the risk of infection and the pain caused by reinstallation of tubes. Traditionally, healthcare workers fix these tubes with pins, adhesive tape, and securement devices. Fixation with pins is less firm and tends to fail due to looseness or slippage. Adhesive tape can be ineffective in fixation when tied too loose and can cause pressure ulcers when tied too tight. Securement devices are hard to apply and are not reusable so they have to be entirely replaced when wound care, dressing change or repositioning is required. Therefore, the traditional affixing means are unsatisfying in terms of operational convenience and fixation performance.

As attempts to solve the problems, some medical-tube affixing devices have been developed. According to existing technical literature, some example of the known medical-tube affixing devices are China Patent Publication No. CN218589449U titled “Kidney internal medicine deep vein temporary hemodialysis catheter fixing device,” China Patent Publication No. CN216963267U titled “Portable medical catheter fixing clamp,” Taiwan Patent No. TWI747549B titled “Nasogastric tube fixing device” that have been granted to the inventor of the present invention, China Patent Publication No. CN213347378U titled “Oxygen tube capable of preventing skin from being damaged and gastric catheter fixing frame,” Taiwan Patent Publication No. TWM546227U titled “Securing and receiving device of gastrostomy tube,” China Patent Publication No. CN218793557U titled “Closed drainage tube clamping device for thoracic surgery” among others. However, none of these prior-art devices can fix multiple tubes while providing convenience in adjusting the device to fit tubes of different sizes and in repositioning the device, and thus none of these prior-art devices can effectively improving working efficiency of healthcare workers.

### SUMMARY OF THE INVENTION

To address the shortcomings of the existing medical-tube affixing devices, the inventor has conducted extensive research and experiments and finally achieve substantive improvements over the prior art.

The present invention provides a medical-tube affixing device, which comprises: an adhesive member having one side adhering to a patient; a base, being fixed to a reverse side of the adhesive member; and a clipping assembly, being movably mounted on the base and being configured to be operated to hold a plurality of medical tubes installed on the body of the patient.

In another embodiment of the present invention, the adhesive member adheres to the patient at one side thereof, and the base has a U-shaped body that is fixed to a reverse side of the adhesive member.

In another embodiment of the present invention, the base defines a slide space formed as a dovetail mortise, two retaining portions that are slender and raised, and two first pivot holes, wherein the two retaining portions are located at a bottom of the slide space, and the first pivot holes are located at two diagonally opposite free corners of the base remote from the retaining portions, in which a first slider is shaped as a dovetail tenon matching the dovetail-mortise-shaped slide space of the base.

In another embodiment of the present invention, a first movable fastener is U-shaped.

In another embodiment of the present invention, a first curved arm and a second curved arm each have a flexible, C-shaped body, and a channel is formed as a through hole with a trapezoid cross-section.

In another embodiment of the present invention, a second slider has a trapezoid body.

In another embodiment of the present invention, the channel of the first slider has two stopping portions in the channel, wherein the stopping portion are configured to engage with two stoppers raised from a bottom of the second slider.

The primary objective of the present invention, a medical-tube affixing device, is to enable a healthcare worker to attach or detach multiple medical tubes to or from a patient easily. The medical-tube affixing device can be adjusted to fit the medical tubes of different sizes and can be repositioned easily, thereby improving working efficiency.

### BRIEF DESCRIPTION OF THE DRAWINGS

FIG. 1 is a perspective view of the present invention.

FIG. 2 is an exploded of the present invention.

FIG. 3 is a cross-sectional, operational view of the present invention.

FIG. 4 is another cross-sectional, operational view of the present invention.

FIG. 5 is a further cross-sectional, operational view of the present invention.

FIG. 6 is an applied view of the present invention.

### DETAILED DESCRIPTION OF THE INVENTION

For further illustrating the means and functions by which the present invention achieves the certain objectives, the following description, in conjunction with the accompanying drawings and preferred embodiments, is set forth as below to illustrate the implement, structure, features and effects of the subject matter of the present invention.

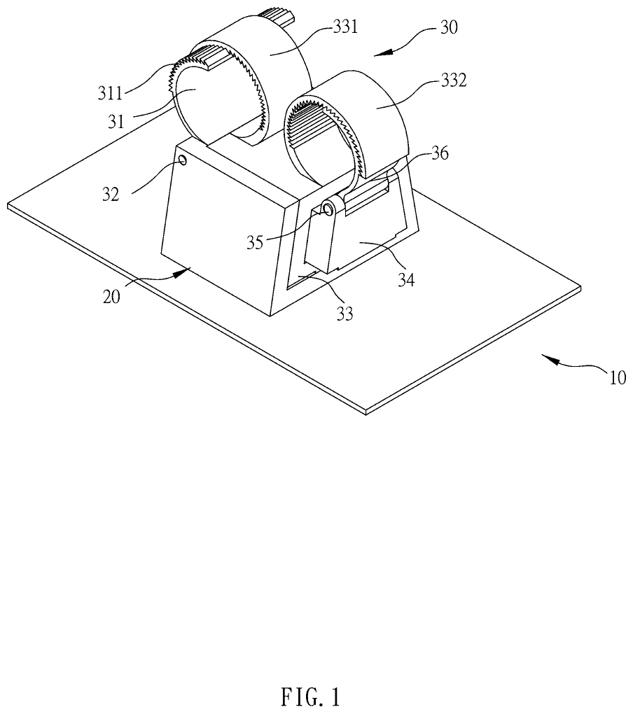

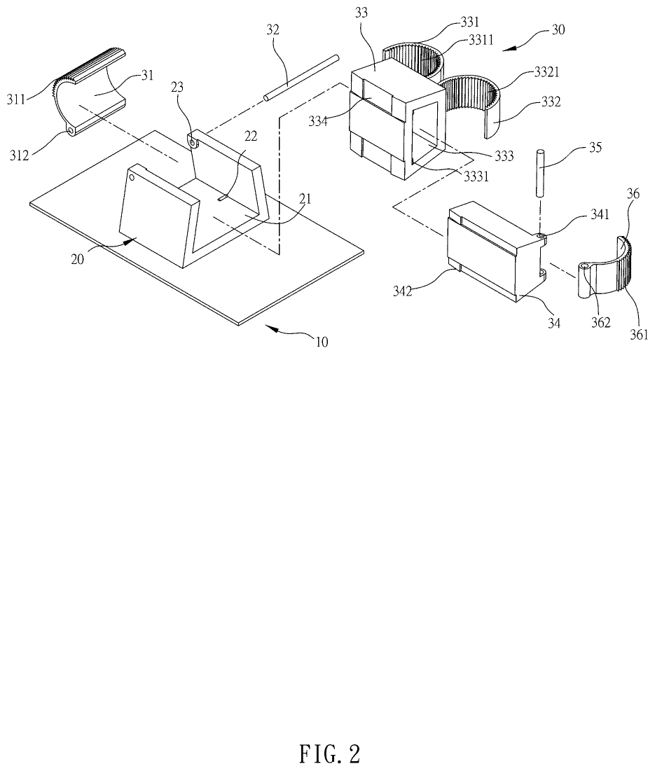

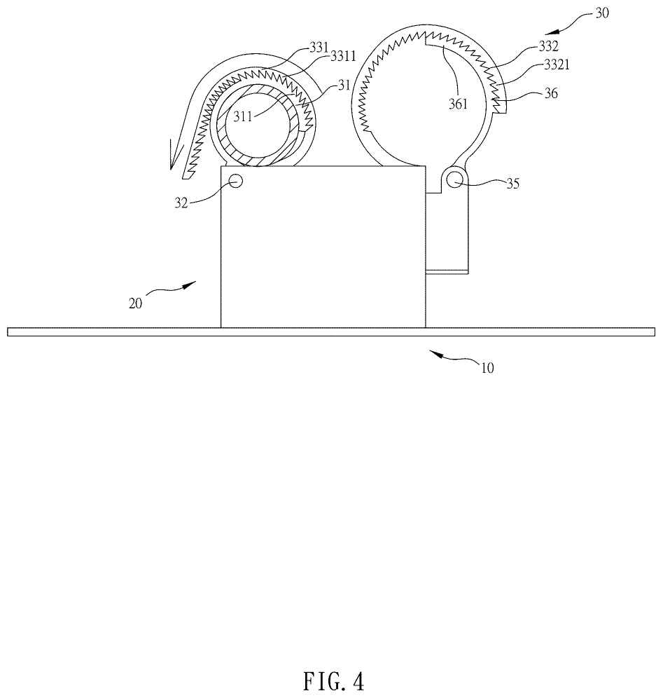

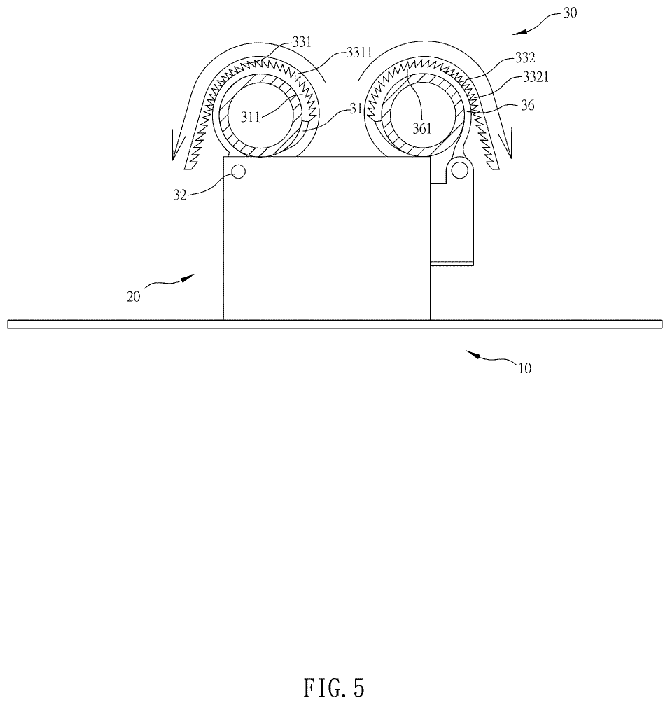

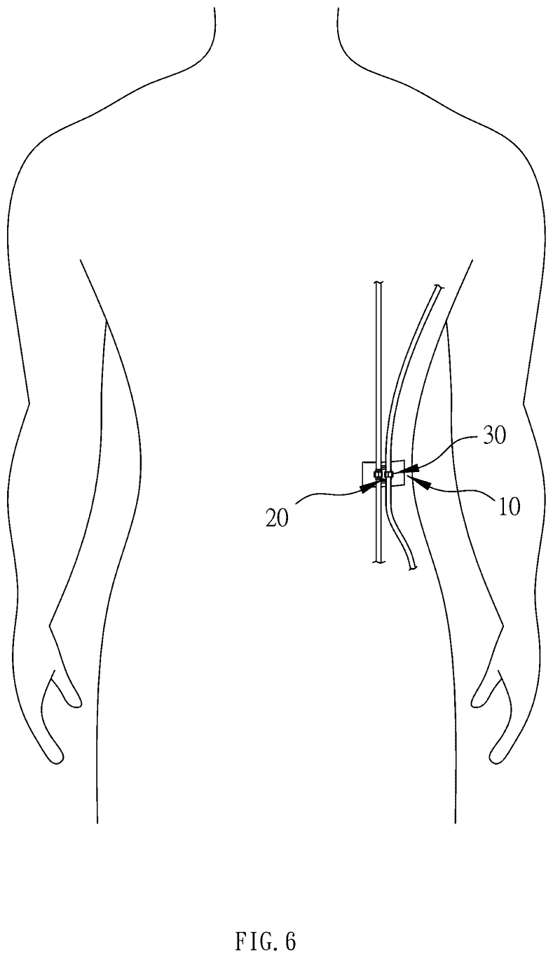

As shown in FIG. 1 through FIG. 14, the present invention provides a medical-tube affixing device for a patient to use. The medical-tube affixing device comprises: an adhesive member 10, a base 20, and a clipping assembly 30. The adhesive member 10 has its one side adhering to the patient. The base 20 is U-shaped base and fixed to a reverse side of the adhesive member 10. The base 20 defines a slide space 21 in the form of a dovetail mortise. The base 20 has two slender, raised retaining portions 22 and two first pivot holes 23. The retaining portions 22 are located at a middle portion of a bottom of the slide space 21, and the first pivot holes 23 are located at two diagonally opposite free corners of the base 20 remote from the retaining portions 22. The clipping assembly 30 includes a first movable fastener 31, a first pivot 32, a first slider 33, a second slider 34, a second pivot 35, and a second movable fastener 36. The first movable fastener 31 is pivotably installed in the slide space 21 at the first pivot holes 23. The first movable fastener 31 is U-shaped and faces the slide space 21 with its concave surface. The first movable fastener 31 has an outward-facing, convex surface provided with a plurality of transversely extending first external teeth 311 near one end of the first movable fastener 31. The first movable fastener 31 further has a first through hole 312 passing through its end remote from the first external teeth 311. The first through hole 312 is locationally corresponding to the first pivot holes 23 in the slide space 21. The first pivot 32 is pivotably received in the first pivot holes 23 of the base 20 and the first through hole 312 of the first movable fastener 31, so that the first movable fastener 31 is swingably mounted on the base 20. The first slider 33 is shaped as a dovetail tenon matching the dovetail-mortise-shaped slide space 21 of the base 20. The first slider 33 is provided with a flexible, C-shaped first curved arm 331, a flexible, C-shaped second curved arm 332, a channel 333 formed as a through hole with a trapezoid cross-section, and a bottom 334. The first curved arm 331 and the second curved arm 332 are located at a middle portion of a free side of the first slider 33. The first curved arm 331 and the second curved arm 332 have their concave, inner surfaces facing the first slider 33. The inner surfaces of the first curved arm 331 and the second curved arm 332 are each formed with a plurality of transversely extending first internal teeth 3311 and second internal teeth 3321. The first internal teeth 3311 of the first curved arm 331 are configured to engage with the first external teeth 311 of the first movable fastener 31. The first slider 33 is slidably fit in the slide space 21 of the base 20 and has its bottom 334 retained by the two retaining portions 22 of the base 20 so that it can slide out of the slide space 21 of the base 20 to a limited extent without separating from the base 20 completely. The channel 333 has two stopping portions 3331 raised from its inner walls near one end of the channel 333. The second slider 34 has a trapezoid body and is slidably fit in the channel 333 of the first slider 33. The second slider 34 has two second pivot holes 341 formed at two free corners of is upper surface. The two second pivot holes 341 are configured to receive a second pivot 35. The second slider 34 has its bottom provided with two raised stoppers 342 to be retained by the raised stopping portions 3331 in the channel 333 of the first slider 33. The second movable fastener 36 is pivotably attached to the second slider 34 by the second pivot 35 passing through the two second pivot holes 341 of the second slider 34. The second movable fastener 36 is U-shaped and has its concave surface facing the slide space 21. The second movable fastener 36 has its convex, outer side formed with a plurality of transversely extending second external teeth 361 near one end of the second movable fastener 36, and further has a second through hole 362 passing through its end remote from the second external teeth 361. The second through hole 362 are configured to align with the two second pivot holes 341 of the second slider 34, so that the second pivot 35 when passing through the second through holes 362 and the second pivot holes 341 of the second slider 34 can swingably attach the second movable fastener 36 to the second slider 34. The second internal teeth 3321 of the second curved arm 332 are configured to engage with the second external teeth 361 of the second movable fastener 36.

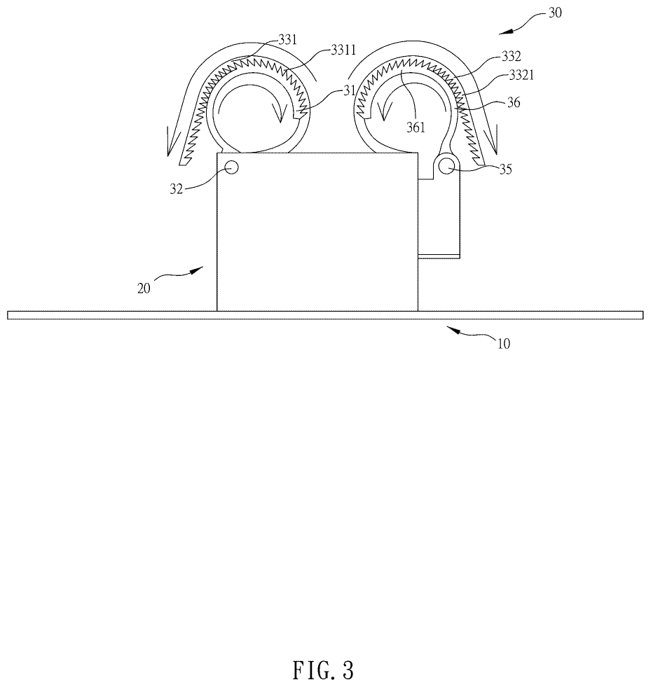

Please refer to FIG. 3 through FIG. 6 for operation of the disclosed device. In use, the adhesive side of the adhesive member 10 can be adhesively attached to the patient. Then a medical tube A installed on the body of a patient can be placed at an opening between the first internal teeth 3311 of the first curved arm 331 and the first external teeth 311 of the first movable fastener 31 that are now separated from each other for tube installation. Then the first internal teeth 3311 of the first curved arm 331 and the first external teeth 311 of the first movable fastener 31 can be drawn together to engage with each other. The first slider 33 can slide in the slide space 21 of the base 20 toward the first movable fastener 31. The first internal teeth 3311 of the first curved arm 331 and the first external teeth 311 of the first movable fastener 31 when engaging with each other jointly define an enclosure for receiving the medical tube A. The first internal teeth 3311 of the first curved arm 331 and the first external teeth 311 of the first movable fastener 31 after engaging with each other can be further adjusted to fittingly receive the medical tube A, thereby facilitating operation and improving working efficiency. If there is another medical tube B installed on the body of the patient, the medical tube B may be placed between the second internal teeth 3321 of the second curved arm 332 and the second external teeth 361 of the second movable fastener 36, so that the second internal teeth 3321 of the second curved arm 332 work with the second external teeth 361 of the second movable fastener 36 to hold the medical tube B. The second internal teeth 3321 of the second curved arm 332 are configured to engage with the second external teeth 361 of the second movable fastener 36, and the second slider 34 can slide in the channel 333 of the first slider 33 toward the second curved arm 332. The second internal teeth 3321 of the second curved arm 332 and the second external teeth 361 of the second movable fastener 36 when engaging with each other jointly define another adjustable enclosure for fittingly receiving the medical tube B, thereby facilitating operation and improving working efficiency. With the configuration and operation described previously, a healthcare worker can efficiently affix multiple medical tubes (such as A and B) to a patient and hold the tubes in position with the convenience in adjusting the clipping assembly 30 to fit the sizes of the used tubes and repositioning the base 20.

Removal of the medical tube A from the disclosed device can be easily achieved by pulling the first curved arm 331 and the first movable fastener 31 apart to separate the first internal teeth 3311 and the first external teeth 311, and sliding the first slider 33 into the slide space 21 of the base 20. Similarly, removal of the medical tube B can be easily achieved by pulling the second curved arm 332 and the second movable fastener 36 apart to separate the second internal teeth 3321 and the second external teeth 361, and sliding the second slider 34 out of the channel 333 of the first slider 33. With use of the disclosed medical-tube affixing device, medical tubes can be easily and firmly attached to/detached from and positioned and/or repositioned with respect to a patient, thereby improving working efficiency of healthcare workers.

With the configuration described previously, the embodiment of the present invention has the following benefits. The disclosed medical-tube affixing device enables a healthcare worker to affix multiple medical tubes to a patient with the adhesive member 10 easily and firmly.

In use of the disclosed medical-tube affixing device, the adhesive side of the adhesive member 10 can be adhesively attached to the patient. Then a medical tube A installed on the body of a patient can be placed at an opening between the first internal teeth 3311 of the first curved arm 331 and the first external teeth 311 of the first movable fastener 31 that are now separated from each other for tube installation. Then the first internal teeth 3311 of the first curved arm 331 and the first external teeth 311 of the first movable fastener 31 can be drawn together to engage with each other. The first slider 33 can slide in the slide space 21 of the base 20 toward the first movable fastener 31. The first internal teeth 3311 of the first curved arm 331 and the first external teeth 311 of the first movable fastener 31 when engaging with each other jointly define an enclosure for receiving the medical tube A. The first internal teeth 3311 of the first curved arm 331 and the first external teeth 311 of the first movable fastener 31 after engaging with each other can be further adjusted to fittingly receive the medical tube A, thereby facilitating operation and improving working efficiency. If there is another medical tube B installed on the body of the patient, the medical tube B may be placed between the second internal teeth 3321 of the second curved arm 332 and the second external teeth 361 of the second movable fastener 36, so that the second internal teeth 3321 of the second curved arm 332 work with the second external teeth 361 of the second movable fastener 36 to hold the medical tube B. The second internal teeth 3321 of the second curved arm 332 are configured to engage with the second external teeth 361 of the second movable fastener 36, and the second slider 34 can slide in the channel 333 of the first slider 33 toward the second curved arm 332. The second internal teeth 3321 of the second curved arm 332 and the second external teeth 361 of the second movable fastener 36 when engaging with each other jointly define another adjustable enclosure for fittingly receiving the medical tube B, thereby facilitating operation and improving working efficiency. With the configuration and operation described previously, a healthcare worker can efficiently affix multiple medical tubes (such as A and B) to a patient and hold the tubes in position with the convenience in adjusting the clipping assembly 30 to fit the sizes of the used tubes and repositioning the base 20.

Removal of the medical tube A from the disclosed device can be easily achieved by pulling the first curved arm 331 and the first movable fastener 31 apart to separate the first internal teeth 3311 and the first external teeth 311, and sliding the first slider 33 into the slide space 21 of the base 20. Similarly, removal of the medical tube B can be easily achieved by pulling the second curved arm 332 and the second movable fastener 36 apart to separate the second internal teeth 3321 and the second external teeth 361, and sliding the second slider 34 out of the channel 333 of the first slider 33. With use of the disclosed medical-tube affixing device, medical tubes can be easily and firmly attached to/detached from and positioned and/or repositioned with respect to a patient, thereby improving working efficiency of healthcare workers.

What is claimed is:

1. A medical-tube affixing device, comprising an adhesive member to adhere to a patient and thereby affixing medical tubes to the patient, and the medical-tube affixing device being characterized in further comprising:
a base, being fixed to the adhesive member and defining therein a slide space and two first pivot holes, wherein the first pivot holes are formed at two free corners of the base; and
a clipping assembly, including a first movable fastener, a first pivot, a first slider, a second slider, a second pivot, and a second movable fastener, the first movable fastener being pivotably installed in the slide space at the first pivot holes, the first movable fastener facing the slide space, the first movable fastener having an outer surface formed with a plurality of transversely extending first external teeth and being formed with a first through hole passing through its end remote from the first external teeth, the first through hole being locationally corresponding to the first pivot holes at the slide space, the first pivot passing through one of the first pivot holes, the first through hole of the first movable fastener, and the other of the first pivot holes to swingably attach the first movable fastener to the base;
the first slider being shaped to match the slide space of the base, the first slider being provided with a first curved arm, a second curved arm, a channel, and a bottom, the first curved arm and the second curved arm being located at a middle portion of a free side of the first slider, the first curved arm and the second curved arm extending and facing the first slider, the first curved arm and the second curved arm having inner surfaces thereof provided with a plurality of transversely extending first internal teeth and a plurality of second internal teeth, the first internal teeth of the first curved arm and the first external teeth of the first movable fastener being configured to engage with each other, the first slider being slidably received in the slide space of the base and having the bottom retained by two retaining portions of the base so the first slider is allowed to slide out of the slide space of the base to a limited extent;
the second slider, being slidably received in the channel of the first slider and having two second pivot holes formed at two free corners of an upper surface thereof, the two second pivot holes being configured to receive the second pivot;
the second movable fastener, being pivotably installed at the two second pivot holes of the second slider, the second movable fastener being U-shaped and facing the slide space, the second movable fastener having an outer surface formed with a plurality of transversely extending second external teeth, and having a second through hole passing through an end thereof remote from the second external teeth, the second through hole being aligned with the two second pivot holes of the second slider, the second pivot passing through one of the second pivot holes of the second slider, the second through hole, and the other of the second pivot holes of the second slider to swingably attach the second movable fastener to the second slider, the second internal teeth of the second curved arm and the second external teeth of the second movable fastener being configured to engage with each other.

2. The medical-tube affixing device of claim 1, being characterized in that the adhesive member adheres to the patient at one side thereof, and the base has a U-shaped body that is fixed to a reverse side of the adhesive member.

3. The medical-tube affixing device of claim 1, being characterized in that the slide space of the base is shaped as a dovetail mortise, and the two retaining portions that are slender and raised, and the two first pivot holes, wherein the two retaining portions are located at a bottom of the slide space, and the first pivot holes are located at two diagonally opposite free corners of the base remote from the retaining portions, in which the first slider is shaped as a dovetail tenon matching the dovetail-mortise-shaped slide space of the base.

4. The medical-tube affixing device of claim 1, being characterized in that the first movable fastener is U-shaped.

5. The medical-tube affixing device of claim 1, being characterized in that the first curved arm and the second curved arm each have a flexible, C-shaped body, and the channel is formed as a through hole with a trapezoid cross-section.

6. The medical-tube affixing device of claim 1, being characterized in that the second slider has a trapezoid body.

7. The medical-tube affixing device of claim 1, being characterized in that the channel of the first slider has two stopping portions raised from inner walls of the channel and near one end of the channel, wherein the stopping portions are configured to engage with two stoppers raised from a bottom of the second slider.
